(19) United States
(12) Patent Application Publication
(10) Pub. No.: US 2011/0059178 A1
(43) Pub. Date: Mar. 10, 2011

(54) Tissue Engineered Meniscus Repair Composition

(75) Inventors: Eric J. Semler, Piscataway, NJ (US); Arthur A. Gertzman, Flemington, NJ (US); Yen-Chen Huang, East Brunswick, NJ (US); Judith Yannariello-Brown, Somerset, NJ (US); Katherine Gomes Truncale, Hillsborough, NJ (US)

(73) Assignee: MUSCULOSKELETAL TRANSPLANT FOUNDATION INC., Edison, NJ (US)

(21) Appl. No.: 12/875,733

(22) Filed: Sep. 3, 2010

Related U.S. Application Data
(60) Provisional application No. 61/240,392, filed on Sep. 8, 2009.

(51) Int. Cl. A61K 9/14; A61F 2/08; A61K 35/34; A61K 35/12; A61P 21/00

(52) U.S. Cl. 424/489; 623/14.12; 424/548; 424/93.7

(57) ABSTRACT

A meniscus repair composition for application to a meniscus defect site to promote growth of new tissue at the meniscus defect site is provided. The composition comprises: from about 10 to about 50 percent by weight of allograft meniscus particles having an average particle size of from about 10 μm to about 500 μm; a carrier selected from the group consisting of sodium hyaluronate, gelatin, collagen, polyethylene glycol, glycerin, carboxymethylcellulose, dextrose, blood derivatives, aqueous solutions thereof, and mixtures thereof; and a curing agent. The curing agent may be the carrier where the carrier is cross-linkable. When introduced to a defect site in a meniscus and cured, the composition will not flow away from the defect site, and the composition is non-adhering to the defect site after it is cured.

### CROSS REFERENCE TO RELATED APPLICATIONS

This application claims priority to U.S. Provisional Application No. 61/240,392, filed Sep. 8, 2009. This Provisional application is incorporated by reference herein, in its entirety and for all purposes.

### TECHNICAL FIELD

The present invention relates generally to the repair and treatment of meniscal injuries. In particular, the present invention relates to a composition and/or an implant comprising the composition wherein the composition comprises allogeneic meniscal tissue and is capable of generating repair tissue at a site of injury to a patient's meniscus once the composition is introduced to the site of injury.

### BACKGROUND

Various publications, including patents, published applications, technical articles and scholarly articles are cited throughout the specification. Each of these cited publications is incorporated by reference herein, in its entirety and for all purposes.

The meniscus plays an important role in load transmission, shock absorption and knee joint stability. Injuries to the meniscus cause pain, disability and damage to the articular cartilage on the femoral and tibial surfaces, leading to development of degenerative changes and osteoarthritis. The meniscus is a dimorphic tissue that consists of two distinctly different tissues, namely the so called “red zone” (vascular) and the so called “white zone” (avascular) tissues.

The red zone, located at the meniscal periphery closest to a vascular blood supply, contains primarily cells that are morphologically fibroblastic. Additionally, the red zone contains a much lesser amount of extra cellular matrix mass than the white zone. Unfortunately, meniscal tears are common in young individuals, usually as a result of sports-related activities, as well as in the older population suffering from degenerative joint diseases. Due to the proximity of the blood supply, lesions, tears and injuries (generally referred to herein as “defects”) in the red zone of the meniscus heal much more rapidly than those occurring in the white zone. Debridement and suturing of the red zone lesions or tears can usually fully restore meniscal function to the red zone, including the restoration of the red zone collagen fibrillar network.

The injuries in the white zone of the meniscus, on the other hand, are currently almost completely untreatable. The white zone itself has no blood supply and is not even located in the proximity of the blood supply. The white zone contains cells that look like chondrocytes typically observed in the articular cartilage, however, the ratio of the extra cellular matrix to cells in the white zone is 10× that of the extra cellular matrix found in the articular cartilage. It is well known that the articular cartilage also does not have any blood supply, that the injuries in the articular cartilage are very difficult to treat, and if they heal at all the ensuing cartilage is an inferior cartilage, called fibrocartilage, rather than normal healthy hyaline cartilage. In this regard the white zone of the meniscus resembles the articular cartilage.

Meniscal defects, particularly those in the white zone, seriously impair the lifestyle of a patient. They can result in altered knee joint function, pain and permanent damage to the adjacent articular cartilage. Due to the avascular nature of the inner white zone region of the meniscus, as described above, a significant number of meniscal lesions or tears do not heal spontaneously. Left untreated, these lesions and tears can propagate into larger defects that exacerbate cartilage damage and the function of the knee.

Early treatments for meniscal injuries typically involve partial or total meniscectomy. This approach frequently results in accelerated cartilage degeneration due to decreased joint contact area and the resultant rise in contact stress. For example, removal of only 15-34% of the meniscus can produce a 350% increase in contact stress. See, e.g., Seedhon B, Hargreaves, D: Transmission of the load in the knee joint with special references to the role of the menisci: II. Experimental results, discussion, and conclusions. Engineering in Med., 8:220 (1979). Therefore, preservation of meniscal tissue and successful lesion repair are the goals of most current treatment methods for meniscal injury.

Currently, a meniscal transplantation is one of the available treatment options for patients whose injury, such as a meniscal tear, is severe and complex. Fresh-frozen allograft menisci have been shown to successfully attach to and heal the recipient periphery in experimental models. Studies have also shown evidence of repopulation of the allograft with host-derived cells. The clinical studies show that 71% of meniscal transplants result in complete healing at 8 months post operation. Despite these positive results, issues with availability of allograft tissue, tissue rejection, disease transmission and a lack of long-term data have limited the use of this approach.

Other types of meniscal repair include suturing a torn gap and stapling or employing pins to reapproximate the torn edges. Although typically successful at mending a torn meniscus, these procedures have significant limitations. For example, sutures and pins are typically polymeric materials that comprise polyalphahydroxy acids such as, for example, poly(glycolic acid) and poly(lactic acid). Such polymers are susceptible to degradation, however, to produce their organic acid monomers which may cause bone dissolution.

Yet another method of repairing a meniscus is to glue the torn tissue together with an adhesive such as, for example, the adhesive disclosed in international patent application Publication No. WO 2006/058215 to Kusanagi et al. Adhesives are difficult to work with in that they are unforgiving once applied so the surgeon has little time to manipulate the adhesive at the injury site. Moreover, the adhesives will adhere the torn edges of a meniscal tear together, but the adhesive material per se remains in the site and prevents the regrowth of new, biologically preferred regenerated tissue (e.g., either new meniscal tissue or less desirable fibrocartilage or fibrous “scar” tissue.

Tissue regeneration is recognized as an alternative way to repair a damaged meniscus. For example, international patent application Publication No. WO 2006/064025 to Pastorello et al. discloses providing a polymer matrix comprising a polymer of hyaluronic acid which purportedly induces the repair of damaged meniscal fibrocartilage by providing intercommunicating pores where cells can colonize and proliferate. In addition to the polymer matrix comprising a polymer of hyaluronic acid, WO 2006/064025 relies on a second supporting three-dimensional matrix comprising polymeric fibers to provide the requisite mechanical strength. Although the polymer matrices introduced by a hyaluronic acid does offer a three dimensional matrix space for cells to enter and grow the desired repair tissue, the use of hyaluronic acid is problematic in that it is rapidly metabolized by the patient and will not remain in place long enough for the complex healing to occur. The addition of polymeric fibers will slow the metabolic decay but not prevent the hyaluronic acid matrix from physically breaking down and again precluding the growth of proper repair tissue.

In view of the foregoing, there is a need in the art for a composition for repairing an injured meniscus and regenerating tissue at the damaged site that does not suffer from the above-mentioned drawbacks.

### SUMMARY

The present invention provides compositions for repairing an injured meniscus and regenerating tissue at the damaged site, and methods of repairing an injured meniscus by regenerating tissue by employing such compositions as disclosed below in multiple embodiments.

In one aspect, the present invention provides a meniscus repair composition for application to a meniscus defect site to promote growth of new tissue at the meniscus defect site, the composition comprising: a) from about 10 to about 50 percent by weight of allograft meniscus particles having an average particle size of from about 10 μm to about 500 μm; b) a carrier selected from the group consisting of sodium hyaluronate, gelatin, collagen, chitosan, alginate, polyethylene glycol, glycerin, carboxymethylcellulose, dextrose, blood derivatives, aqueous solutions thereof, and mixtures thereof; and c) a curing agent, wherein the curing agent may be the carrier when the carrier is cross-linkable, wherein the composition, when introduced to a defect site in a meniscus and cured, will not flow away from the defect site, and wherein the composition is non-adhering to the defect site after it is cured.

In some aspects, the invention provides methods for repairing a knee meniscus injury. Generally, the methods comprise administering a composition such as those described and/or exemplified herein to a site at least proximal or adjacent to the injury. The composition may be administered directly to the injury. Once administered, the composition may be secured at a desired location. Where the composition comprises a suture, the injury may be sutured closed with the suture, with the effect that the composition is secured in place by nature of the suturing. Once administered to a meniscus injury, the compositions facilitate the growth of new meniscus tissue at the meniscus injury. The compositions may facilitate one or more of blood vessel formation, fibrochondrocyte production, cell infiltration, or formation of three-dimensional meniscus tissue at the meniscus injury. The methods generally facilitate healing of the injury.

### BRIEF DESCRIPTION OF SEVERAL VIEWS OF THE DRAWINGS

The invention is best understood from the following detailed description when read in connection with the accompanying figures. It is emphasized that, according to common practice, the various features of the figures are not to scale. On the contrary, the dimensions of the various features are arbitrarily expanded or reduced for clarity. Included are the following figures:

FIG. 1 illustrates the anatomy of a knee joint.

FIG. 1A shows a top planar view taken at a cross section of FIG. 1 along the line 1A-1A.

FIG. 2 illustrates an embodiment of the present invention.

FIG. 3 illustrates an embodiment of the present invention.

### DETAILED DESCRIPTION

The present invention generally relates to compositions and methods for repairing an injured meniscus and regenerating tissue at the damaged site. In particular, a composition and method increase the rate of meniscus repair and induce the formation of more normal (i.e., endogenous-type) meniscal tissue than has been commonly observed heretofore. A meniscual repair composition can enhance or otherwise facilitate the body's natural tissue repair processes to repair any injury to a meniscus. The compositions are suitable for repairing any injury, including trauma, mechanical injury, surgical incision, surgical resection, tissue wear, tissue degeneration, or any other injury to the meniscus, from whatever source of the injury.

A meniscal repair composition can induce meniscus repair of avascular tears and fill the defect with meniscus-like tissue. Moreover, a composition and method are useful for repairing and regenerating meniscal tissue which has been removed by partial or complete meniscectomy. A composition and method can enhance blood vessel formation, produce fibrochondrocytes, induce cellular infiltration into the composition, induce cellular proliferation, and produce cellular and spatial organization to form a three-dimensional meniscus tissue.

As used herein, the singular forms “a,” “an,” and “the” include plural referents unless expressly stated otherwise.

The term “implant” is used to refer to tissue, compositions of the invention or cells (xenogeneic or allogeneic) which may be introduced into the body of a patient to replace or supplement the structure or function of the endogenous tissue.

The terms “autologous” and “autograft” refer to tissue or cells which originate with or are derived from the recipient, whereas the terms “allogeneic” and “allograft” refer to cells and tissue which originate with or are derived from a donor of the same species as the recipient. The terms “xenogeneic” and “xenograft” refer to cells or tissue which originate with or are derived from a species other than that of the recipient.

The term “exposing” refers to soaking the tissue in a fluid comprising the treatment agent for a period of time sufficient to treat the tissue. The soaking may be performed by, but is not limited to, incubation, swirling, immersion, mixing, or vortexing.

The term “paste” or “injectable paste” refers to a mixture of minced or milled pretreated allograft meniscus in a biocomposite carrier having a viscosity which is less than and is less rigid than a mixture of minced or milled pretreated allograft meniscus in a biocompatible carrier referred to by the term “putty” and contains less meniscus by weight than putty.

A “hydrogel” refers to a semisolid composition constituting a substantial amount of water, and in which polymers or mixtures thereof are dissolved or dispersed. The hydrogels may be physically or chemically cross linked. Hydrogels according to the invention may be components of gels, putties, and/or pastes.

The term “dehydration” broadly refers to any method that removes the water from the tissue without denaturing the tissue. This includes, but is not limited to, processes such as, by way of example and not limitation, lyophilization, vacuum drying, air drying, or solvent-based drying such as, by way of example and not limitation, exposing the tissue to various alcohol-based solutions.

The term “tissue” is used in the general sense herein to mean any transplantable or implantable tissue, the survivability of which is improved by the methods described herein upon implantation. In particular, the overall durability and longevity of the implant are improved, and host-immune system mediated responses are substantially eliminated. The tissue herein includes but is not limited to meniscus; ligaments; basal membrane; dermis; tendons; cartilage tissue; tubular tissue such as, by way of example and not limitation, arterial tissue and vein tissue; heart valve tissue; demineralized bone tissue; tissues used to construct heart valves such as, by way of example and not limitation, dura mater and pericardium tissue; transparent tissue such as, by way of example and not limitation, cornea and lens tissue; membrane-like tissue such as, by way of example and not limitation, porcine pericardium; bovine pericardium; porcine intestine tissue and lung tissue, more specifically porcine sub-mucosa tissue; bladder tissue; human tissue that is generated and discarded during human childbirth, e.g., human placenta and umbilical cord tissue generated during child birth; amniotic membrane tissue; and the like.

The term “polymerizable” denotes that molecules have the capacity to form additional covalent bonds resulting in monomer interlinking to oligomer or polymer formation, for example, molecules that contain carbon-carbon double bonds of acrylate-type molecules. Such polymerization is characteristically initiated by free-radical formation, for example, resulting from photon absorption of certain dyes and chemical compounds to ultimately produce free-radicals. The term polymerizable is also applicable to compounds which can undergo condensation polymerization and form a linear or cross linked polymer.

Compositions

One embodiment provides a meniscus repair composition for application to a meniscus defect site to promote growth of new tissue at the meniscus defect site, the composition comprises: a) from about 10 to about 50 percent by weight of allograft meniscus particles having an average particle size of from about 10 μm to about 500 μm; b) a carrier selected from the group consisting of sodium hyaluronate, gelatin, collagen, chitosan, alginate, polyethylene glycol, glycerin, carboxymethylcellulose, dextrose, blood derivatives, aqueous solutions thereof, and mixtures thereof; and c) a curing agent, wherein the curing agent may be the carrier when the carrier is cross-linkable, wherein the composition, when introduced to a defect site in a meniscus and cured, will not flow away from the defect site, and wherein the composition is non-adhering to the defect site after it is cured.

The composition comprises allograft meniscus particles. The allograft meniscus particles function in several ways. For example, the allograft meniscus particles function to provide a matrix (i.e., a physical three-dimensional environment sufficient to act as a scaffold for infiltrating cells to support tissue growth). New meniscus tissue may grow or at least collagen fibrous tissue can fill the space using the meniscal tissue as a matrix upon which the new meniscal tissue will grow. This, in effect, allows for the regeneration of a functional tissue filling a gap or tear in a patient's meniscus. The allograft meniscus particles also function to provide the biochemical cues to initiate a healing response from cells that have either infiltrated the matrix from surrounding host tissue and bleeding bone or from cells that have been added initially to the composition.

The allograft meniscus particles of the composition preferably have an average particle size of from about 10 μm to about 500 μm, more preferably from about 10 μm to about 250 μm, and most preferably from about 10 μm to about 100 μm. In one embodiment, the allograft meniscus particles have a size (e.g., at least one dimension) within a range of from about 10 microns to about 210 microns (i.e., from about 0.01 mm to about 0.21 mm). Alternatively, the allograft meniscus particles may have a size (i.e., the aforesaid at least one dimension) that is within a range of from about 10 microns to about 120 microns (i.e., from about 0.01 mm to about 0.12 mm). The at least one dimension of the allograft meniscus particles may alternatively be less than or equal to 212 microns; within a range of from about 5 microns to about 212 microns; within a range of from about 6 microns to about 10 microns; less than or equal to 5 microns; less than or equal to 10 microns; or less than or equal to 100 microns. In another embodiment, the at least one dimension of most of the particles is less than 106 microns. In yet another embodiment, the at least one dimension of the allograft meniscus particles has a mean and/or median value in the range of between 10 microns and 200 microns. The small size of the allograft meniscus particles can facilitate the increased exposure of, or release of, various growth factors due to the increased aggregate surface area of the particulate allograft meniscus used, and can increase the capacity of the surrounding and infiltrating cells to attach to the allograft meniscus particles.

The allograft meniscus particles can be a mixture of red and white zone allograft meniscus, substantially all red zone allograft meniscus particles, or substantially all white zone allograft meniscus particles. Separation of the allograft meniscus particles into substantially red zone particles or substantially white zone particles yields compositions that benefit from the inherent endogenous chemical composition of each respective anatomical zone. For example, the biochemical composition of a human meniscus comprises a mixture of endogenous growth factors such as, for example, transforming growth factor beta (TGF-β), vascular endothelial growth factor (VEGF), bone morphogenic protein-2 (BMP-2), insulin-like growth factor 1 (IGF-1), thrombin peptide 508 (TP 508), and nel-like molecule 1 (Nell-1). Vascular endothelial growth factor, however, is more prevalent in the vascular-containing red zone of the meniscus. Thus, a composition whose allograft meniscal particles comprise substantially all red zone allograft particles is particularly useful in repairing defects in the red zone because they can deliver higher doses of growth factors endogenous to the red zone.

The allograft meniscus particles are preferably prepared by a process that cleans, sterilizes, lyophilizes, and grinds the lyophilized meniscus tissue. In one exemplary embodiment, the allograft meniscus particles are prepared by a process comprising the steps of: disinfecting an allograft meniscus; cutting the allograft meniscus into multiple pieces; lyophilizing the allograft meniscus pieces; grinding the pieces at a temperature of below about 4° C. to achieve ground allograft meniscus particles having the average particle size of from about 10 μm to about 500 μm; and separating unwanted particles by sieving the ground allograft meniscus particles through a sieve having an appropriately sized mesh.

Disinfecting an allograft meniscus may comprise exposing the allograft meniscus to multiple solutions such as, for example, a solution of an oxidizing agent such as, for example, hydrogen peroxide, H₂O₂, an alcohol solution, and optionally a solution of a non-ionic surfactant. In addition to such solutions, it is preferred to also employ frequent intermittent purified water washes. Exposing the allograft meniscus to such solutions is preferably carried out under suitable agitation at a temperature of from below about 34° C.

The oxidizing agent is provided in an aqueous solution and, thus, contains water. The oxidizing agent portion of the solution can be from about 0.5 to about 30 percent by weight of the solution, preferably from about 1 to about 10 percent by weight of the solution and, more preferably, from about 3 to about 5 percent by weight of the solution. Suitable oxidizing agents include, but are not limited to, hydrogen peroxide, periodic acid, peracetic acid, sodium iodate, sodium hypochlorite, and mixtures thereof. Hydrogen peroxide is the preferred oxidizing agent.

The alcohol solution is an aqueous solution and, thus, contains water. The alcohol portion of the solution can be from about 10 to about 90 percent by weight of the solution, preferably from about 20 to about 80 percent by weight of the solution and, more preferably, from about 30 to about 70 percent by weight of the solution. Suitable alcohols include, but are not limited to, ethanol, propanol, iso-propanol, hexanol, and mixtures thereof. A mixture of ethanol and iso-propanol is preferred.

When present, the non-ionic surfactant is provided in an aqueous solution and, thus, contains water. The non-ionic surfactant portion of the solution can be from about 0.01 to about 10 percent by weight of the solution, preferably from about 0.01 to about 3 percent by weight of the solution and, more preferably, from about 0.10 to about 1 percent by weight of the solution. Suitable non-ionic surfactants include, but are not limited to, Triton® X-100 (Union Carbide Corp., NY), Tween® 80 (ICI Americas, Inc., DE), N,N-dimethyldodecylamino-N-oxide, octylglucoside, polyoxyethylene (PEG) alcohols, polyoxyethylene-p-t-octylphenol, polyoxyethylene nonylphenol, polyoxyethylene sorbitol esters, polyoxy-propylene-polyoxyethylene esters, p-iso-octylpolyoxy-ethylene-phen-ol formaldehyde polymer, and mixtures thereof. Triton® X-100 is the preferred non-ionic surfactant.

In some embodiments, the disinfecting step further comprises exposing the allograft meniscus or meniscus pieces to an antibiotic solution. Preferred antibiotics include, for example, gentamicin, amphotericin B, primaxin, erythromycin, bacitracin, neomycin, penicillin, polymyxin B, tetracycline, viomycin, chloromycetin and streptomycin, cefazolin, ampicillin, azactam, tobramycin, triclosan, clindamycin, and mixtures thereof.

In some embodiments, the disinfecting step further comprises exposing the allograft meniscus or meniscus pieces to a buffered saline solution to ensure removal of the above-identified process solutions. Preferably, the saline solution is buffered at a pH of about 6.5 to about 7.8 and, more preferably from about 7.2 to about 7.4.

Preferably, the disinfecting step functions to remove antigenic elements, residual cellular debris, and lipids from the allograft meniscus or allograft meniscus pieces. Exposure time to each of the above-identified solutions, if employed, can be anywhere from 1 minute to 24 hours, preferably, from 1 hour to 8 hours, and more preferably from 3 to 5 hours. The order of the steps of the disinfecting process are not critical to the invention; however, exemplary processes are disclosed in U.S. patent application Publication No. 2004/0037735, which is incorporated herein by reference.

The process includes the step of cutting the allograft meniscus into multiple pieces. This step can be performed before or after the above-described disinfecting step. Any suitable sterile cutting means known in the art such as, for example, a scissor or scalpel, can be employed to cut the allograft into multiple pieces. Preferably the allograft meniscus is cut into pieces of from about 85 to about 300 μm of irregularly-shaped polygonal particles.

The process includes the step of lyophilizing the allograft meniscus pieces. Those skilled in the art will appreciate that lyophilization is a freeze-drying process in which water is sublimed from the composition after it is frozen. The particular advantage associated with the lyophilization process is that biological materials can be dried without elevated temperatures, thereby eliminating the adverse thermal effects. An exemplary lyophilization process includes an initial shelf temperature of from about −20° C. to about −55° C., and preferably about −40° C. for about 4 hours, with the temperature raised to +35° C. for about 28 hours, with the last 29 hours being under a vacuum of about 350 mTorr.

The process of the present invention includes the step of grinding the pieces of allograft meniscus at a temperature of from less than about 4° C. to achieve ground allograft meniscus particles having the average particle size of from about 10 μm to about 500 μm, more preferably from about 10 μm to about 250 μm, and most preferably from about 10 μm to about 100 μm. The allograft meniscus particles according to the present invention are preferably cryogenically ground (i.e., below −185° C.) to achieve such desired particle size. In some embodiments of the present invention, the ground lyophilized meniscus tissue is sieved through an appropriately sized mesh screen to achieve the desired average particle size. Employment of a sieve is an optional component. The average particle size of the allograft meniscus particles is determined by methods well known to the skilled artisan such as, for example, by capturing and analyzing digital images of particles with a microscope and an image analysis program such as ImagePro Plus® (Media Cybernetics Inc., MD).

Such processes described above are preferably performed in a manner which ensures the efficacy of the processed tissue for introduction into a human patient. Accordingly, the processes are preferably performed in a sterile environment such as, for example, a Class 10 clean room. More preferably, the processed tissue is, at some point prior to introduction into a human patient, further sterilized by exposure to radiation such as, for example, gamma or electron-beam radiation at a dose of from about 3 to about 30 kiloGreys.

The compositions include a carrier selected from the group consisting of hyaluronic acid, gelatin, collagen, polyethylene glycol, glycerin, carboxymethylcellulose, dextrose, blood derivatives, aqueous solutions thereof, and mixtures thereof. The primary role of the carrier is to serve as a delivery vehicle. The bulk viscosity of the carrier achieves the design goal of either flowability or good handling properties of the composition by balancing the molecular weight and concentration of the carrier used in the formulation. For example, to achieve a specific bulk viscosity, one would typically use a lower concentration of a very high molecular weight carrier, wherein a higher concentration of a lower molecular weight carrier would be required to achieve the same bulk viscosity.

A preferred carrier for use in compositions is hyaluronic acid (HA). Hyaluronic acid encompasses sodium hyaluronate, hyaluronic acid, pharmaceutically acceptable salts of hyaluronic acid, derivatives of hyaluronic acid and pharmaceutically acceptable salts of hyaluronic acid derivatives and mixtures thereof. Preferably, the HA has a molecular weight of about 7.0×10⁵ to 3.0×10⁶ Daltons. The HA is typically employed at a concentration of from 0.5 to 5% by weight in water or phosphate buffered saline to achieve the bulk viscosity required for an injectable paste composition or from 1 to 5% by weight in water or phosphate buffered saline to achieve the bulk viscosity required for the paste or putty composition.

The carrier may be a hydrogel-forming polymer or a non-hydrogel forming substance. Exemplary hydrogel-forming polymers suitable for use as carriers include, but are not limited to, sodium hyaluronate having a molecular weight of from about 6.6×10⁵ to about 2.6×10⁶ Daltons and its derivatives; and carboxymethylcellulose (CMC) having a molecular weight of from about 20,000 to about 40,000 Daltons. Examples of non-hydrogel forming substances that are suitable for use as a carrier include collagen, gelatin, and blood derivatives such as, for example, platelet rich plasma (PRP), platelets, venous and/or arterial blood.

The term “aqueous mixtures thereof” as used in connection with the carrier refers to an aqueous solution component of the carrier. An aqueous solution component of the carrier includes an aqueous buffer solution. In preferred embodiments, the carrier includes an aqueous buffer solution to make the composition more pH compatible with the biological environment of a human patient and the fluids that may come into contact with the composition once implanted therein. The natural condition for blood plasma is at a pH of 7.3 to 7.4 (reference, Principles of Biochemistry, Chapters 34 & 35; White, Handler and Smith, McGraw Hill, N.Y., 1964). At very slight changes in pH, blood cells will shift their equilibrium of hemoglobin. This hemoglobin concentration will change over the small pH range of 7.3 to 7.7 (White et al. p. 664). Moreover, at significantly lower pH values in the acidic range, protein molecules will degrade. Thus, it is important to maintain any surgical implant which is in intimate contact with blood at a biocompatible condition of from about pH 7.2 to about 7.4.

To achieve a pH of from about pH 7.2 to about 7.4, the composition preferably employs a phosphate buffer based on an aqueous system of the two phosphate anions, HPO₄⁻² and H₂PO₄⁻¹. The pH is adjusted to the physiologic range of 6.8 to 7.4 pH or preferably 7.2 to 7.4 pH by using either or both of dibasic sodium phosphate or monobasic sodium phosphate and adjusting the solution with saline, i.e., a sodium chloride solution. The sodium chloride is chosen instead of only water so as to control the final osmolality of the composition to preclude dehydration of the surrounding cells.

A typical viscosity range for injectable paste compositions of the present invention is from about 2,000 cps to about 15,000 cps at ambient temperature. A typical viscosity range for putty compositions of the present invention is from about 120,000 cps to about 270,000 cps at ambient temperature. Viscosity may be determined according to any suitable technique in the art.

In certain embodiments, the carrier is a blood derivative such as, for example, platelet rich plasma (PRP). As used herein, the term “platelet rich plasma” refers to a preparation consisting of platelets concentrated in a limited volume of blood plasma. The repair response of musculoskeletal tissues generally starts with the formation of a blood clot and degranulation of platelets, which releases growth factors and cytokines at the site. This microenvironment results in chemotaxis of inflammatory cells as well as the activation and proliferation of local progenitor cells. In most cases, fibroblastic scar tissue is formed. In some settings, however, such as in a fracture callus, these conditions can also facilitate the formation of new tissue. The following endogenous growth factors can be found in the environment of a blood clot: transforming growth factor beta (TGF-β); platelet-derived growth factor (PDGF); insulin-like growth factor (IGF); vascular endothelial growth factors (VEGF); epidermal growth factor (EGF); and fibroblast growth factor-2 (FGF-2).

Preferably, the platelet rich plasma is autologous PRP, i.e., it is the patient's own plasma and platelets that have been isolated and concentrated either before or during surgery. Autologous PRP is preferred because it contains a biologically active mixture of growth factors without the potential for an immune response. Additional (i.e., non-inherent) growth factors may also be added to the PRP as described in more detail below.

It is an important aspect that the compositions once introduced to a defective site on a patient's meniscus must remain at the defective site and not be washed away by the flowing blood and other fluids brought to the site by the healing mechanism. Accordingly, the compositions include a curing agent to effect a curing or cross-linking or hardening of the composition at the appropriate time (either before or after introduction into a patient's defective meniscus tissue such that the composition, when cured, will not flow away from the defect site). The curing agent can be a chemical curing agents or a photo-oxidative curing agent. A chemical curing agent can be any suitable known curing agent including, but not limited to, formaldehyde, glutaraldehyde, dimethyl suberimidate, carbodiimides, multi-functional epoxides, succinimidyls, genipin, tyramine, poly(glycidyl ether), diisocyanates, acyl azide, tris(hydroxymethyl)phosphine, ascorbate-copper, ribose, glucose, and glucose-lysine. In one embodiment, the chemical curing agent is an aldehyde.

In embodiments where the curing agent is a photo-oxidative curing agent, the curing is typically initiated by exposure to radiation such as, for example, UV radiation. As used herein, the term “photo-oxidative curing agent” refers to a compound that, when activated by exposure to radiation, will cause transfer of electrons or hydrogen atoms and thereby oxidize the carrier in the presence of oxygen. Exemplary photo-oxidative curing agents suitable for use in the compositions of the present invention include, for example, methylene blue, methylene green, rose bengal, riboflavin, proflavin, fluorescein, eosin, and pyridoxal-5-phosphate.

In embodiments where a photo-oxidative curing agent is employed in a composition, the composition can be irradiated at an appropriate time, depending on the particular embodiment. For example, in embodiments where the composition is an injectable paste, it can be irradiated once injected into a defective site on a patient's meniscus. Exposure to radiation at least causes local curing (i.e., polymerization) to the surface exposed to the radiation thereby forming a “skin” thereon and, preferably, such exposure initiates polymerization throughout the composition thereby causing it to harden. If the composition is a putty, the composition can be formed into a shape and then irradiated. Such photo-oxidation is typically achieved with a UV radiation source (i.e., a UV light); however, in some embodiments, it could also be achieved using incandescent, white light or fluorescent light, i.e., visible light, or that portion of light in the visible range that is absorbed by the curing agent.

The intensity of the light employed, and the length of time required to cross-link or cure the composition will vary depending upon several factors. These include: (1) the type and amount of photo-oxidative curing agent; (2) the thickness of the composition; (3) the distance between the composition and the irradiation source; and (4) the type and intensity of the light source. For instance, exposure time may vary from as little as a few seconds up to as much as about 10 hours. With regard to the intensity of the light, one or more lights may be used of intensity preferably ranging up to about 150 watts, preferably held at a distance from about 0.1 cm to 12 cm from the sample surface.

Depending on the type of curing agent employed, one of ordinary skill in the art will appreciate when the curing agent can be mixed with the bulk of the allograft meniscus composition. For example, in embodiments where the curing agent is a photo-oxidative curing agent, the photo-oxidative curing agent can typically be intimately mixed with the other components of the composition without concern for premature curing so the compositions can be stored for an indefinite period of time as long as the storage container is opaque to light. In embodiments where the curing agent is a chemical curing agent, the curing agent is typically kept separate from the other components of the composition until a surgical procedure to repair a defective meniscus. Separation of the chemical curing agent from the bulk of the composition can be achieved in several ways. For example, the composition can be provided, prior to use, in a kit wherein the chemical curing agent is included in a container separate from the other mixed components followed by mixing prior to use. Such composition can also be provided in a dual syringe mixing device such as, for example, the device disclosed in U.S. Pat. No. 7,018,382, which is incorporated herein by reference. In such mixing devices, the chemical curing agent can be separated from the bulk of the composition until the composition is ready for use.

In certain embodiments, the curing agent is the carrier itself where the carrier is cross-linkable without the aid of a separate and distinct cross-linking agent. Examples of such embodiments include compositions where the carrier comprises collagen and/or gelatin or chitosan or alginate, and, thus, can be cured, for example, by the application of energy such as, for example, heat.

Additives that are beneficial to tissue growth may be added to the compositions at any stage of the mixing process. Such additives include living cells and cell elements such as chondrocytes, red blood cells, white blood cells, platelets, blood plasma, bone marrow cells, mesenchymal stem cells, pluripotent cells, osteoblasts, osteoclasts, and fibroblasts, epithelial cells, and endothelial cells. These cells or cell elements or combinations of the same are typically present at a concentration of 10⁵ to 10⁸ per cc of carrier and are added into the composition at the time of surgery.

Growth factor additives can also be added to the compositions either at the time of packaging or at surgery, depending on the stability of the growth factor. Such growth factors include, but are not limited to transforming growth factor beta (TGF-β), insulin growth factor (IGF-1); platelet derived growth factor (PDGF), vascular endothelial growth factor (VEGF), fibroblast growth factor (FGF) (numbers 1 to 23 and, in particular, numbers 2, 4 and 9), bone morphogenic factors (BPM 2, 4, 7, 9 and 14), osteopontin, growth hormones such as somatotropin cellular attractants and attachment agents.

Any number of medically useful substances can also be added to the compositions such as, for example, antiviral agents (such as those effective against HIV and hepatitis), amino acids, peptides, vitamins, co-factors for protein synthesis, hormones, endocrine tissue or tissue fragments, synthesizers, enzymes (such as collagenase, peptidases, oxidases), polymer cell scaffolds with parenchymal cells, angiogenic drugs and polymeric carriers containing such drugs, collagen lattices, biocompatible surface active agents, antigenic agents, cytoskeletal agents, cartilage, and cartilage fragments.

The compositions are not adhesive compositions and, thus, are non-adhering to surfaces such as, for example, the surfaces of a meniscal defect site, after the composition is cured. Accordingly, it is preferred that the compositions, when cured, are secured to the defect site by a securing mechanism such as, for example, staples, sutures, or a biological glue. Thus, the compositions can comprise one or more sutures, staples, or biological glues. Suitable biological glue can be found commercially, such as for example, TISSEEL® (Baxter Int'l, Inc., DE) or TISSUCOL (fibrin based adhesive; Immuno AG, Austria), Adhesive Protein (Sigma Chemical, USA), and Dow Corning Medical Adhesive B (Dow Corning, USA).

The compositions are typically made by adding the appropriate amount of allograft meniscus particles to a carrier and mixing under shear. The carrier may or may not have additional components such as, for example, BMP, mesenchymal stem cells, and a curing agent (except a chemical curing agent unless the composition is mixed just prior to introduction into a meniscal defect site). For a flowable/injectable paste, from about 2 to about 25 weight percent of the allograft meniscus particles can be added. For a malleable putty, from about 10 to about 50 weight percent of the allograft meniscus particles can be added. The malleable property permits the surgeon to shape the quantity of meniscal putty or paste to exactly fit the surgical defect. Manipulation of the “lump” of meniscal putty may be done without it sticking to the gloves of the surgeon, behaving somewhat like a wet clay used in sculpting.

Those of ordinary skill in the art would readily appreciate that multiple formulation variables are considered in achieving a specific compositional viscosity such as, for example, molecular weight of the carrier, pH, mixing temperature, degree of shear force during mixing, and particle size. Each of these variables can be adjusted to achieve the desired flow properties without undue experimentation.

Preformed Meniscal Implants

Another embodiment provides a rigid or semi-rigid preformed meniscal implant comprising any of the above-described compositions. The shape of the preformed implant can be, for example, a wedge, a crescent shape, a disc, a block, or a nugget. Such shapes can be further trimmed by a surgeon to fit a prepared torn space and sutured or glued in place.

To make such embodiments, any of the compositions as described above is mixed and placed into a mold. The composition is then cross-linked or cured. For example, in embodiments where a photo-oxidative curing agent is employed, the composition can be irradiated with UV light once in the mold and cured therein. In embodiments where a chemical curing agent is employed, the composition can be mixed with the chemical curing agent and introduced into a mold within which the composition will harden. In compositions where the carrier itself is cross-linkable such as, for example, aqueous gelatin or collagen (and, thus, no separate and distinct curing agent is needed), the composition can be mixed and introduced into a mold followed by lyophilization.

In other embodiments, the allograft meniscus paste/putty can be incorporated into either a decellularized soft tissue or a demineralized cancellous, cortical, or corticocancellous bone scaffold. As used herein, the term “scaffold” refers to a supporting framework of such tissues.

In embodiments where the scaffold is demineralized cancellous bone, the cancellous bone may originate from an allograft proximal or distal femur, proximal or distal tibia, proximal humerus, talus, calceneus, patella, or ileum. Cancellous tissue is first processed into blocks and then milled into the desired shapes such as, for example, a wedge or a crescent shape. The cancellous bone is then typically substantially demineralized in dilute acid until the bone contains less than 0.5% wt/wt residual calcium. Subsequently, the resultant tissue form is predominantly Type I collagen, which is sponge-like in nature with an elastic quality. Following decalcification, the tissue is further cleaned and may also be treated so that the cancellous tissue is non-osteoinductive. This inactivation of inherent osteoinductivity may be accomplished via chemical (e.g., hydrogen peroxide) or thermal treatment or by high energy irradiation. Once demineralized, the allograft meniscus particle paste or putty can be introduced into the sponge-like structure and cured at the appropriate time. The resulting implant can be glued or sutured into a defective site of a patient's meniscus.

In embodiments where the scaffold is decellularized soft tissue, the tissue is preferably allograft human soft tissue which has been previously taken from a human donor and frozen for later use. The soft tissue which is envisioned as being used is facia, dermis, cartilage, pericardium, human valves and veins, tendons and ligaments. The following is an exemplary process for preparing the soft tissue scaffold for use.

To prepare the soft tissue scaffold, the soft tissue which has been previously obtained from a donor and frozen is taken from the freezer and thawed. Prior to processing, tissue is inspected for damage (holes or tears) and distinctive features (moles, warts, tattoos) which are removed using a scalpel. Tissue is inspected for hair and the same is removed using forceps. A visual inspection is performed to ensure the tissue has uniform thickness. Any region of non-uniformity or visibly low thickness is removed. To identify the orientation (dermal or epidermal side) of tissue such as skin, the skin is positioned such that the epidermis faces the processor and an incision is cut into the upper left corner of each piece of tissue to indicate the epidermal side.

After thawing, the soft tissue is processed and decellularized. Dermis, for example, is decellularized using a 1M Sodium Chloride (NaCl) solution in a tissue flask and agitated at a speed of 65 RPM on an orbital shaker for a minimum of 12 hours, up to a maximum of 48 hours. The epidermal layer of the skin is removed at this time and rinsed with sterile water. The remaining dermis is placed in the tissue flask filled with sterile water and agitated on an orbital shaker for 15 minutes. The sterile water is refreshed and the rinse procedure is repeated one more times for a total of two rinses. Once the rinse is complete, the water is replaced with 0.1% Triton® X-100 solution and agitated on the orbital shaker for a minimum of 24 hours, up to a maximum of 48 hours. The dermis is then rinsed with sterile water, placed in the tissue flask filled with sterile water, and agitated on the orbital shaker at 65 RPM for 15 minutes. The sterile water is refreshed and the rinse procedure is repeated a minimum of 5 more times. A residual detergent test is performed on the rinsate to ensure the detergent has been adequately removed. The two steps utilizing 1M NaCl and 0.1% Triton soaks may be combined.

The decellularized dermis is subjected to sterilization in a solution containing peracetic acid, alcohol, propylene glycol, and water and soaked and agitated at 65 RPM under vacuum for a minimum of 4 hours, up to a maximum of 8 hours. The dermis undergoes a rinse series followed with agitation at 65 RPM under vacuum: two 5-minute rinses, followed by two 10-minute rinses, followed by two 15-minute rinses for a total of 6 rinses. After the last rinse, the residual test is performed on the rinsate to ensure that the peracetic acid has been adequately removed.

Other tissue such as fascia, cartilage, pericardium, tendons and ligaments is soaked in antibiotic soak for 1.5 to 24 hours and rinsed with sterile phosphate buffered saline. If desired the other tissue can also be soaked in 1M NaCl for 1.5 to 48 hours, rinsed with sterile water a plurality of times, then soaked in 0.1% Triton® X-100 for 4 to 48 hours and rinsed with sterile water until a minimal residual amount of detergent is reached. The tissue is soaked in peracetic acid in vacuum for 4 hours and rinsed with sterile water from 6 to 9 times until a minimum residual amount of acid is tested.

The sterilized tissue is cut to finished size. The fascia and dermis can be perforated with holes about 1.2 mm in diameter spaced from each other 2 to 3 mm. The tissue is dipped in 70% ethanol and 30% water and packaged. Compositions can be introduced into the perforated tissue either before packaging or by the surgeon in the operating room prior to implantation into a patient.

Shaped Meniscal Implant with Supporting Intra-Tibial Load Plate

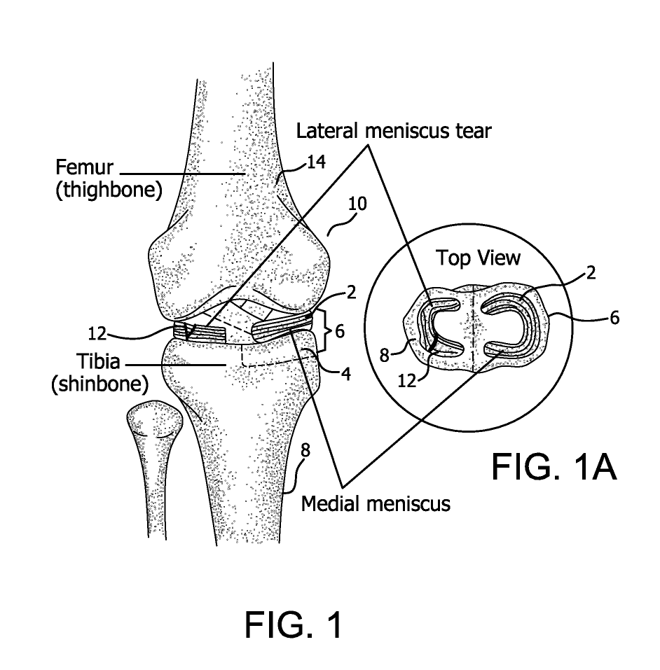

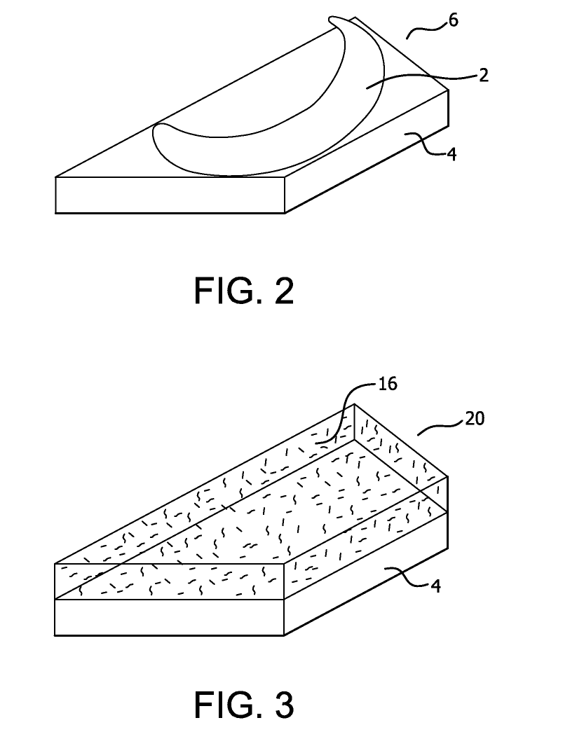

The present invention also addresses conditions where a patient's meniscus has experienced a higher degree of damage and a more invasive surgery needs to be performed to correct the defect such as, for example, a partial or total meniscectomy. Accordingly, another embodiment provides a meniscus-shaped implant having a supporting intra-tibial load bearing plate where either the medial or lateral meniscus needs to be replaced in its entirety. Referring to FIGS. 1 & 1A, a human knee joint assembly 10 is depicted that includes a shaped meniscal implant 6 according to the present invention, a lateral meniscus 12, a tibia 8, and a femur 14. Also referring to FIG. 2, the shaped meniscal implant 6 comprises a preformed implant 2 and a supporting load plate 4, which is attached to the tibia 8. The implant 2 as shown in FIGS. 1 and 2 is a medial meniscus implant; however, an implant as described herein can also be a lateral meniscus implant.

Preformed implant 2 is an implant comprising any of the above-described preformed compositions and preferably has the crescent shape of a healthy meniscus (see FIG. 1A). Preformed implant 2 functions as a replacement meniscus and is affixed to supporting load plate 4 by any suitable mechanism known in the art such as, for example, biological glue.

Referring to FIG. 3, a shaped meniscal implant 6 can be formed from a composite block 20. Composite block 20 comprises supporting load plate 4 and an unshaped layer 16 that comprises a composition as described herein. Unshaped layer 16 can comprise, for example, a layer of demineralized cancellous allograft bone comprising a composition as described above. Unshaped layer 16 can be shaped by a surgeon in the operating room with suitable tools such as, for example, a scalpel. As shown in FIG. 3, unshaped layer 16 is attached to supporting load plate 4; however, unshaped layer 16 and supporting load plate 4 can be provided in a kit as separate layers so the surgeon can shape unshaped layer 16 into a preformed implant prior to attaching the preformed implant to supporting load plate 4.

Supporting load plate 4 functions to support the load on the meniscus during the healing period and after recovery. Supporting load plate 4 can be machined out of any suitable material such as, for example, cortical or dense cancellous allograft bone (that has been processed to achieve an immunologically neutral state); sterilized ceramic biomaterials such as, for example, tricalcium phosphate, calcium carbonate, calcium hydroxyapatite, sea coral, and bioglass (aluminum silicates); metal alloys such as, for example, titanium metal alloys and stainless steel alloys; and plastics such as, for example, high density polyethylene.

Once the surgeon prepares tibia 8 for receiving the shaped meniscal implant 6, the supporting load plate 4 portion may be secured to tibia 8 by any suitable means known in the art. Such suitable mechanisms include, for example, biological glue and/or physical structures such as, for example, male/female connectors or dove tail slots.

Method for Treatment and Repair of Meniscal Defects

The method of the invention is directed to treatment and repair of the meniscal injuries.

In practice, the surgeon determines the size of the meniscal tear or lesion and the extent of injury. Depending on the size of the tear or lesion, the surgeon decides if the injury will be treated just with a composition either in injectable paste or putty form or if a pre-formed implant needs to be deposited in conjunction with a composition.

In either case, the composition is deposited into the tear or lesion either alone or optionally in the form of a preformed implant that may or may not need further modification prior to implantation. For smaller-size injuries, typically the composition alone—in the form of a putty or injectable paste—is deposited into the meniscal tear or lesion. For larger or complicated injuries, tears or lesions, the preformed implant is typically preferred. The allograft meniscal particle composition may be deposited after the implantation of a scaffold as described above or it may be introduced into the scaffold before its implantation into the tear or lesion. Alternatively, where no scaffold is employed, the preformed implant can be inserted into the defect area and secured by a securing mechanism such as, for example, sutures or application of biological glue.

In both instances, the tears or lesions are filled with the allograft meniscus particle composition in situ during the arthroscopic surgery. If a paste or putty is employed, typically the composition cures either by action of the chemical curing agent or by photoinitiation. Upon curing, the composition preferably fills the gap completely and is attached to the meniscal walls surrounding the tear or lesion by a securing means such as, for example, sutures or application of biological glue. The composition remains there until the tear or lesion closes and heals, typically within several weeks or months. Since the tear or lesion gap is filled, there is no friction between the two sides of the tear or lesion, there is no further deterioration or enlargement of the tear, nor is there an accompanying deterioration of the adjacent articular cartilage.

The surprising and unexpected effect of the compositions and methods of the present invention is that actual meniscus or meniscus-like new tissue will grow in and on the allograft meniscus particles. The patient's adjacent meniscus and/or microvasculature will provide mesenchymal stem cells to the site using the implanted meniscal particles as a matrix to enter the damaged (i.e., defective) space and engineer new meniscal tissue. The mesenchymal stem cells can differentiate their environment and proliferate into meniscal cells filling the gap with regenerated meniscal tissue and providing relief from the preoperative pain experienced by the patient.

Additional objects, advantages, and novel features of these inventions will become apparent to those skilled in the art upon examination of the following examples. The examples are included to more clearly demonstrate the overall nature of the inventions and, thus, are illustrative and not restrictive of the inventions.

### EXAMPLES

### Example 1

### Meniscus Processing

### Generally

Both the lateral and medial meniscus are recovered from the left and right knees of a donor by blunt dissection. At this point, the meniscus may be further dissected into a substantially red zone section and a substantially white zone section. Any residual soft tissue is removed and then each meniscus is subjected to a serious of chemical soaks and rinses. In one embodiment, the meniscal tissue is first soaked in an antibiotic solution containing gentamicin, primaxin and amphotericin B for up to 4 hours at 20° C. to 40° C. under agitation, followed by multiple rinses in a saline buffer. In another embodiment, the tissue is also subsequently soaked in a detergent (such as Polysorbate 80 or Triton® X-100) or dilute acid (such as HCl, acetic acid, or peracetic acid) or base (such as NaOH) to further clean the tissue. Single or multiple soaks may be performed for up to 1 hour at 20-40° C. under agitation. More specifically, the tissue can be soaked in 0.1% Triton® X-100 for 15-30 minutes on a reciprocating or orbital shaker at a temperature of approximately 37° C. Following this soak, the meniscal tissue is rinsed multiple times with a saline buffer to remove Triton residuals prior to further processing.

Subsequently, each meniscus is cut into pieces that are approximately no more than 5 mm by 5 mm with a thickness of no more than 5 mm. Cutting of the meniscus can be performed using a scalpel or with a semi-automated or an automated chopping device. The meniscus strips are then lyophilized to a residual moisture level of less than 6% wt/wt. After dehydration, meniscus strips are then subjected to a pulverization process under liquid nitrogen using a freezer milling device (Spex CertiPrep, Metuchen, N.J.). In one embodiment, the milled pieces are sieved to obtain a particle size of less than 212 microns. In another embodiment, the milled pieces are sieved to a particle size of less than 850 microns. In one embodiment, these particles are then stored in this dehydrated state until reconstitution. In another embodiment, these meniscal particles are then further cleaned by soaking in a detergent, dilute acid or base, or disinfecting agent such as hydrogen peroxide or ethanol under agitation. After additional chemical soaks, saline rinses are performed to remove residuals and then the particles would be again lyophilized to a residual moisture level of less than 6% wt/wt.

For reconstitution, meniscal particles can be mixed with saline or combined with a carrier to create a paste or putty-like formulation. In one example, meniscal particles are reconstituted in saline to a concentration of 25-40% wt/wt. In another example, one (1) gram of the allograft meniscus particles is mixed into 4 grams of an aqueous buffered (pH 7.2) sodium hyaluronate (mol. wt. of about 700,000 Daltons) solution resulting in a 25% (w/w) viscous dispersion which may be delivered from a syringe. When mixed with a either a chemical or UV curing agent, this viscous dispersion can be surgically injected into a defect in a patient's meniscus and cured by the appropriate method. The cured composition may be secured in position by applying a suitable commercial tissue adhesive such as Tisseel® or by suture.

The following are additional examples of curing meniscus pastes.

### Example 1A

### Chemical Crosslinking Via Genipin

For curing by chemical crosslinking, genipin is first reconstituted in phosphate buffered saline (pH 7.4) to a concentration of 0.1-10% (w/v). Dehydrated meniscal particles are then added to the genipin solution at a concentration of 20-40% (w/v) for 1-2 hours at 37° C. under gentle agitation for the crosslinking step. Following crosslinking, the residual genipin is removed from the matrix composition by multiple rinses with phosphate buffered saline.

### Example 1B

### Non-Enzymatic Glycation Via Ribose or Glucose

For curing by non-enzymatic glycation, either a solution of ribose (1-5 mg/mL) or glucose (15-30 mg/mL) is first prepared. Subsequently, dehydrated meniscal particles are added to the sugar solution at a concentration of 20-40% (w/v). Incubation times in the sugar-rich solution may range from several hours to weeks. Subsequently, the glycation reaction is terminated by extensive rinsing with phosphate buffered saline.

### Example 1C

### Thermo-Sensitive Chitosan-Based Hydrogel Carrier

Meniscal particle compositions may be cured by suspending the particles in a thermo-responsive hydrogel that hardens when the temperature rises to near physiologic levels. For example, chitosan hydrogels may be designed to gel near 37° C. Chitosan hydrogels are formed by first dissolving chitosan in 1% acetic acid to form a 3-6% (w/v) solution. Next, the chitosan solution and the meniscal particles in a buffered saline are mixed together at room temperature. After mixing, the temperature is raised to 37° C. and the mixed solution forms a gel after several minutes. The pH of the resulting gel may be modified by the addition of sodium bicarbonate to the mixture prior to gelation.

### Example 1D

### Photochemical Crosslinking Via Riboflavin and UV Irradiation

Riboflavin is a photosensitizing agent that produces free radicals upon exposure to UV light and subsequent crosslinking of collagen fibrils. Riboflavin 5-phosphate is reconstituted in 20% Dextran to a final concentration of 0.1% (w/v). The riboflavin solution is then added to the meniscal particle composition such that the particles are entirely submerged in the solution. Subsequently, the composition is crosslinked by exposing the mixture to UV irradiation with an intensity between 2-4 mW/cm². The residual riboflavin solution is removed from the resulting gel by rinsing with phosphate buffered saline.

### Example 2

Mesical particles having an average particle size of <212 microns are prepared and sterilized as described in Example 1. The particles are dried by lyophilization. The dried particles are added to a solution of gelatin as follows:

| Component | Weight (gr.) | Weight Percent |
|---|---|---|
| Meniscus Particles | 9.00 | 45% |
| Gelatin | 2.34 | 11.7% |
| NaHy (2% solution) | 3.30 | 16.5% |
| Phosphate Buffer | 5.36 | 26.8% |
| (pH = 7.2) | | |
| Total | 20 | 100% |

The resultant solution is poured into a mold in the shape of a rectangular block 5 mm×5 mm×5 mm and then lyophilized to remove almost all of the water content.

The resultant form when removed from the mold is in the shape of a rigid block.

A surgeon can cut the block into a specific shape to fit a specific meniscal defect in a patient. The shaped block softens when re-wetted by exposure to the body fluids of the receiving patient. See, e.g., U.S. Pat. No. 7,045,141, incorporated herein by reference.

### Example 3

A shaped implant as described in Example 2 is affixed to an allograft bone block. The bone block consists of a mineralized cancellous bone in a cylindrical shape. A molded meniscal block in the shape of a meniscal arm (e.g., medial arm of an anatomically intact meniscus) is adhesively adhered to the bone block using a commercially available fibrin-thrombin glue (e.g., Tisseel®). The meniscal block is 5 mm×5 mm×5 mm.

The principles, preferred embodiments and modes of operation of the present inventions have been described in the foregoing specification. The inventions should not be construed as limited, however, to the particular embodiments which have been described above. Instead, the embodiments described here should be regarded as illustrative rather than restrictive. Variations and changes may be made by those skilled in the art without departing from the scope as defined by the claims that follow. It is expressly intended, for example, that all ranges broadly recited in this document include within their scope all narrower ranges which fall within the broader range.

1. A meniscus repair composition comprising:
a) from about 10 percent to about 50 percent by weight of allograft meniscus particles having an average particle size of from about 10 μm to about 500 μm;
b) a carrier selected from the group consisting of: sodium hyaluronate, gelatin, collagen, chitosan, alginate, polyethylene glycol, glycerin, carboxymethylcellulose, dextrose, blood derivatives, aqueous solutions thereof, and mixtures thereof; and
c) a curing agent, wherein the curing agent may be the carrier where the carrier is cross-linkable;
wherein the composition, when administered to a knee meniscus injury and cured, will not flow away from the injury, and wherein the composition is non-adhering to the injury after it is cured.

2. The composition of claim 1, wherein the composition is an injectable paste.

3. The composition of claim 1, wherein the composition is a putty.

4. The composition of claim 1, further comprising a growth factor.

5. The composition of claim 4, wherein the growth factor is an allogenic growth factor.

6. The composition of claim 4, wherein the growth factor is an autologous growth factor.

7. The composition of claim 4, wherein the growth factor comprises one or more of TGF-β, VEGF, BMP-2, IGF-1, Nell-1, and TP 508.

8. The composition of claim 1, further comprising mesenchymal stem cells.

9. The composition of claim 1, wherein the curing agent is a UV curing agent and is initially intimately mixed with the composition, wherein upon exposure to UV radiation the composition will cure at least at the site of the UV exposure.

10. The composition of claim 1, wherein the curing agent is a chemical curing agent and is contained separate from the composition, wherein the chemical curing agent is intimately mixed with the composition at the time that the composition is administered.

11. A shaped meniscal implant produced by a method comprising the steps of:
introducing the composition of claim 1 into a mold;
curing the composition; and
removing the cured composition from the mold.

12. A shaped meniscal implant produced by a method comprising the steps of:
introducing the composition of claim 1 into a mold wherein the carrier is selected from the group consisting of: gelatin, collagen, and a mixture thereof;
lyophilizing the composition; and
removing the lyophilized composition from the mold.

13. A shaped meniscal implant produced by a method comprising the steps:
introducing the composition of claim 1 into a matrix selected from the group consisting of: tendon, dermis, and demineralized cancellous bone; and
curing the composition.

14. A meniscal implant assembly comprising:
the shaped meniscal implant of claim 11, wherein the shape is a crescent shape; and
a supporting intra-tibial load bearing plate affixed to the shaped meniscal implant.

15. The meniscal implant assembly of claim 14, wherein the supporting intra-tibial load bearing plate comprises allograft bone.

16. The meniscal implant assembly of claim 14, wherein the supporting intra-tibial load bearing plate comprises a ceramic material.

17. The meniscal implant assembly of claim 16, wherein the ceramic material is selected from the group consisting of: tricalcium phosphate, calcium carbonate, calcium hydroxyapatite, sea coral, and aluminosilicate bioglass.

18. The meniscal implant assembly of claim 14, wherein the supporting intra-tibial load bearing plate comprises titanium metal.

19. The meniscal implant assembly of claim 14, wherein the supporting intra-tibial load bearing plate comprises demineralized bone.

20. The meniscal implant assembly of claim 14, wherein the supporting intra-tibial load bearing plate is affixed to the shaped meniscal implant by a means selected from the group consisting of: glue, dovetail slots, or mating appendages.

21. A method for repairing a knee meniscus injury, comprising administering a composition according to claim 1 proximal to the injury.

22. The method of claim 21, comprising administering the composition to the injury.

23. The method of claim 21, wherein the composition facilitates the growth of new meniscus tissue at the meniscus injury after administering the composition to the meniscus injury.
